(19) United States
(12) Patent Application Publication
(10) Pub. No.: US 2026/0199132 A1
(43) Pub. Date: Jul. 16, 2026

(54) SYSTEMS, DEVICES, AND METHODS THAT ENABLE ACCESS TO ON-AXIS AND OFF-AXIS TISSUE TARGETS

(71) Applicant: ViaLase, Inc., Aliso Viejo, CA (US)

(72) Inventor: Ferenc Raksi, Mission Viejo, CA (US)

(21) Appl. No.: 19/019,258

(22) Filed: Jan. 13, 2025

(51) Int. Cl. A61F 9/009; A61B 3/10; A61B 3/117; A61F 9/008

(52) U.S. Cl. CPC A61F 9/009; A61B 3/102; A61B 3/117; A61F 2009/00851

(57) ABSTRACT

An adaptable ophthalmic system includes an on-axis system and an off-axis system that couples to and decouples from the on-axis system. The on-axis system has an on-axis-system axis and includes a distal end and optics. The optics are configured to provide an on-axis beam path that extends through the distal end at an angle within an on-axis threshold angle of the on-axis-system axis. The off-axis adapter has an off-axis-adapter axis and includes a distal end and optics. When coupled to the on-axis system, the off-axis adapter optically couples the on-axis beam path with the optics. The optics are configured to optically align the on-axis beam path with an off-axis beam path that extends through the distal end at an angle greater than an off-axis threshold angle from the on-axis-system axis to thereby access off-axis targets.

### TECHNICAL FIELD

The present disclosure relates generally to the field of medical devices and treatment of diseases in ophthalmology, and more particularly to systems, devices, and methods that enable access to on-axis and off-axis tissue targets in the eye, including the irido-corneal angle.

### BACKGROUND

With reference to FIG. 1, the outer tissue layer of the eye 1 includes a sclera 2 that provides the structure of the eye's shape. In front of the sclera 2 is a cornea 3 that is comprised of transparent layers of tissue that allow light to enter the interior of the eye. Inside the eye 1 is a crystalline lens 4 that is connected to the eye by fiber zonules 5, which are connected to the ciliary body 6. Between the crystalline lens 4 and the cornea 3 is an anterior chamber 7 that contains a flowing clear liquid called aqueous humor 8. Encircling the perimeter of the crystalline lens 4 is an iris 9 which forms a pupil around the approximate center of the crystalline lens. The vitreous humor 10 is located between the crystalline lens 4 and the retina 11. Light entering the eye is optically focused through the cornea 3 and crystalline lens.

The corneoscleral junction of the eye, also referred to as the irido-corneal angle 13, is the portion of the anterior chamber 7 at the intersection of the iris 9, the sclera 2, and the cornea 3. The anatomy of the eye 1 at the irido-corneal angle 13 includes a trabecular meshwork. The trabecular meshwork is a fibrous network of tissue layers that encircles the iris 9 within the eye 1. The network of tissue layers that make up the trabecular meshwork are porous and thus present a pathway for the egress of aqueous humor 8 flowing from the anterior chamber 7. This pathway may be referred to herein as an aqueous humor outflow pathway, an aqueous outflow pathway, or simply an outflow pathway.

As an optical system, the eye 1 is represented by an optical model described by idealized centered and rotationally symmetrical surfaces, entrance and exit pupils, and six cardinal points: object and image space focal points, first and second principal planes, and first and second nodal points. Angular directions relative to the human eye are often defined with respect to an optical axis 24, a visual axis 26, a pupillary axis 28 and a line of sight 29 of the eye. The optical axis 24 is the symmetry axis, the line connecting the vertices of the idealized surfaces of the eye. The visual axis 26 connects the foveal center 22 with the first and second nodal points to the object. The line of sight 29 connects the fovea through the exit and entrance pupils to the object. The pupillary axis 28 is normal to the anterior surface of the cornea 3 and is directed to the center of the entrance pupil. These axes of the eye differ from one another only by a few degrees and fall within a range of what is generally referred to as the direction of view.

Different ophthalmic procedures require access to different target tissues of the eye. For example, in procedures for treating corneal conditions, the cornea 3 may need to be accessed for purposes of imaging and surgical treatment, e.g., laser treatment. In procedures for treating cataracts or refractive conditions, target tissues in the anterior segment of the eye, e.g., the crystalline lens 4, the posterior capsule of the lens, the anterior capsule of the lens, the vitreous humor 10 or the retina 11, may need to be accessed for purposes of imaging and surgical treatment. These target tissues, together with the cornea 3, are referred to herein as on-axis targets. In procedures for treating glaucoma, target tissue in the irido-corneal angle 13 may need to be accessed for purposes of imaging and surgical treatment. These target tissues are referred to herein as off-axis targets.

It would be beneficial to have an ophthalmic system that is easily configurable to access either on-axis targets and off-axis targets.

### SUMMARY

The present disclosure relates to an adaptable ophthalmic system that includes an on-axis system and an off-axis system that couples to and decouples from the on-axis system. The on-axis system has an on-axis-system axis and includes a distal end and optics. The optics are configured to provide an on-axis beam path that extends through the distal end at an angle within an on-axis threshold angle of the on-axis-system axis. The off-axis adapter has an off-axis-adapter axis and includes a distal end and optics. When coupled to the on-axis system, the off-axis adapter optically couples the on-axis beam path with the optics. The optics are configured to optically align the on-axis beam path with an off-axis beam path that extends through the distal end at an angle greater than an off-axis threshold angle from the on-axis-system axis to thereby access an off-axis target.

The present disclosure relates to a method of optically accessing an off-axis structure of an eye. The method includes providing an on-axis beam path that extends through a distal end of an on-axis system having an on-axis-system axis. The on-axis beam path is at an angle within an on-axis threshold angle of the on-axis-system axis. The method also includes optically aligning the on-axis beam path with an off-axis beam path that extends through a distal end of an off-axis adapter. The off-axis beam path is at an angle greater than an off-axis threshold angle from the on-axis-system axis and provides access to an off-axis target.

The present disclosure also relates to an off-axis adapter for directing a light beam between a distal end of an on-axis system and a structure distal the on-axis system. The on-axis system has an on-axis system axis and includes optics that are configured to provide an on-axis beam path that extends through the distal end at an angle within an on-axis threshold angle of the on-axis-system axis. The off-axis adapter includes a proximal optical element configured to optically couple to and decouple from the on-axis system; a distal optical element configured to optically couple to and decouple from the structure; and optics between the proximal optical element and the distal optical element. The optics optically align with the on-axis beam path and are configured to convert the on-axis beam path to an off-axis beam path that extends through the distal optical element at an angle greater than an off-axis threshold angle from the on-axis-system axis to thereby access an off-axis target.

The present disclosure further relates to an adaptable ophthalmic system that includes a system having a system axis and an adapter having an adapter axis. The system includes optics coupled to receive a light beam and configured to direct the light beam along a first beam path. The adapter is configured to couple to and decouple from the system. When coupled to the system, the adapter axis and the system axis are substantially coaxially aligned, and optics of the adapter optically couple with the optics of the system to receive the light beam. The optics of the adapter are configured to redirect the light beam to a second beam path that is angularly offset from the first beam path by a threshold angle.

In one configuration, the system is an on-axis system and the adapter is an off-axis adapter. The first beam path is an on-axis beam path that extends through a distal end of the on-axis system, wherein the on-axis beam path is at an angle within an on-axis threshold angle of the system axis. The second beam path is an off-axis beam path that extends through a distal end of the off-axis adapter, wherein the off-axis beam path is at an angle greater than an off-axis threshold angle from the system axis.

In another configuration, the system is an off-axis system and the adapter is an on-axis adapter. The first beam path is an off-axis beam path that extends through a distal end of the off-axis system, wherein the off-axis beam path is at an angle greater than an off-axis threshold angle from the system axis. The second beam path is an on-axis beam path that extends through a distal end of the on-axis adapter, wherein the on-axis beam path is at an angle within an on-axis threshold angle of the system axis.

It is understood that other aspects of apparatuses and methods will become apparent to those skilled in the art from the following detailed description, wherein various aspects of apparatuses and methods are shown and described by way of illustration. As will be realized, these aspects may be implemented in other and different forms and its several details are capable of modification in various other respects. Accordingly, the drawings and detailed description are to be regarded as illustrative in nature and not as restrictive.

### BRIEF DESCRIPTION OF THE DRAWINGS

Various aspects of systems, apparatuses, and methods will now be presented in the detailed description by way of example, and not by way of limitation, with reference to the accompanying drawings, wherein:

FIG. 1 is a sectional schematic illustration of a human eye and its interior anatomical structures, and showing various axes associated with the eye.

FIG. 2 is an illustration of an adaptable ophthalmic system that enables access to off-axis targets by a system configured to only access on-axis tissue targets.

FIG. 3A is a sectional schematic illustration of an on-axis light beam path.

FIG. 3B is a sectional schematic illustration of an off-axis light beam path.

FIG. 4 is a block diagram of an embodiment of an adaptable ophthalmic system for visualizing an eye and/or delivering a treatment to an eye.

FIG. 5A is a schematic illustration of components of the adaptable ophthalmic system of FIG. 4 is a decoupled state, the components including an on-axis system, an off-axis adapter, and an optional patient interface.

FIG. 5B is a schematic illustration of the components of the adaptable ophthalmic system of FIG. 5A is a coupled state.

FIG. 6 is a block diagram of another embodiment of an adaptable ophthalmic system for visualizing an eye and/or delivering a treatment to an eye.

FIG. 7A is a schematic illustration of components of the adaptable ophthalmic system of FIG. 6 is a decoupled state, the components including an on-on-axis system, an off-axis adapter with imaging components, and an optional patient interface.

FIG. 7B is a schematic illustration of the components of the adaptable ophthalmic system of FIG. 7A is a coupled state.

FIG. 8 is a flowchart of a method of enabling access to on-axis and off-axis tissue targets in the eye.

FIG. 9 is an illustration of an adaptable ophthalmic system that enables access to on-axis targets by a system configured to only access off-axis tissue targets.

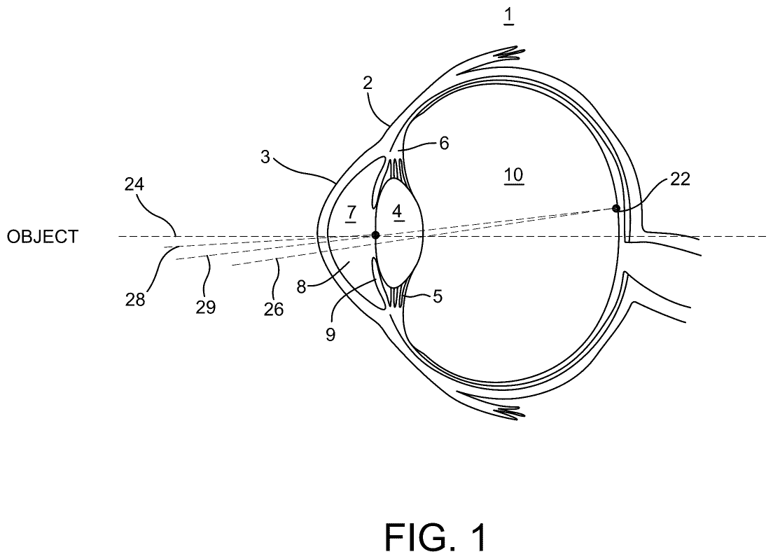

### DETAILED DESCRIPTION

Systems, devices, and methods disclosed herein adapt an ophthalmic system that is configured to only access targets in some segments of the eye to an ophthalmic system that can access targets in other segments of the eye. In some embodiments, an ophthalmic system that only accesses on-axis targets of the eye is adapted into an ophthalmic system that accesses off-axis targets. Thus, an ophthalmic system designed for on-axis surgeries, such as corneal surgeries, e.g., flap pocket, tunnel, keratoplasty, small incision lenticule extraction (SMILE); or cataract surgeries, e.g., capsulotomy, lens fragmentation, and corneal incisions, can be used to perform off-axis surgeries, e.g., glaucoma surgery and trabeculoplasty. In some embodiments, an ophthalmic system that only accesses off-axis targets in an eye is adapted into an ophthalmic system that accesses on-axis targets. Thus, an ophthalmic system designed for off-axis surgeries, such as glaucoma surgery and trabeculoplasty, can be used to perform on-axis surgeries, such as corneal surgeries and or cataract surgeries. Integrating multiple ophthalmic surgical application into one system is a cost-effective equipment in a surgeon's clinic. It allows significant reduction of capital equipment costs and occupied space in the clinic compared to owning different equipment for different applications

### On-Axis to Off-Axis Adaptive System

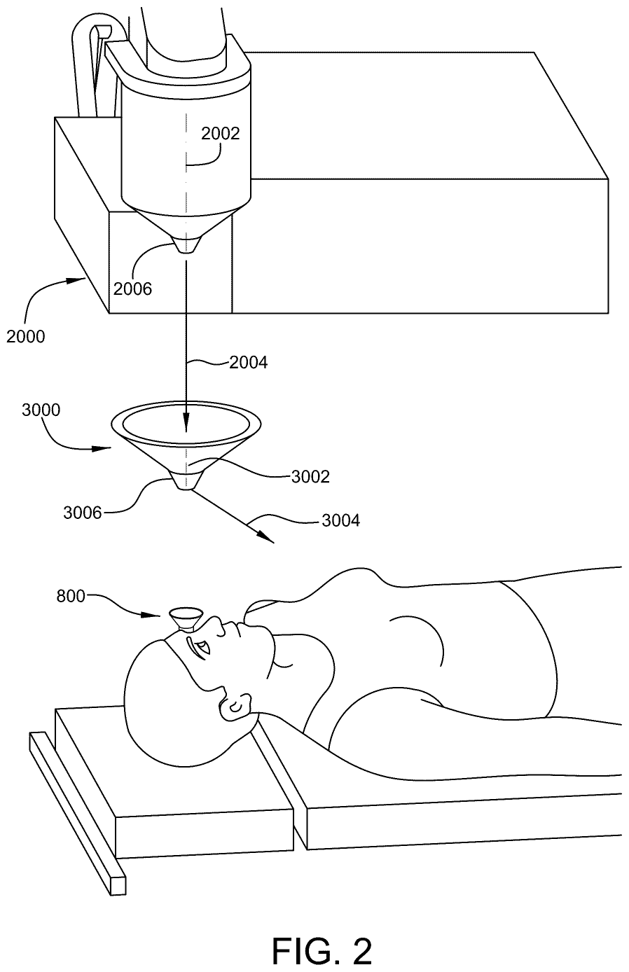

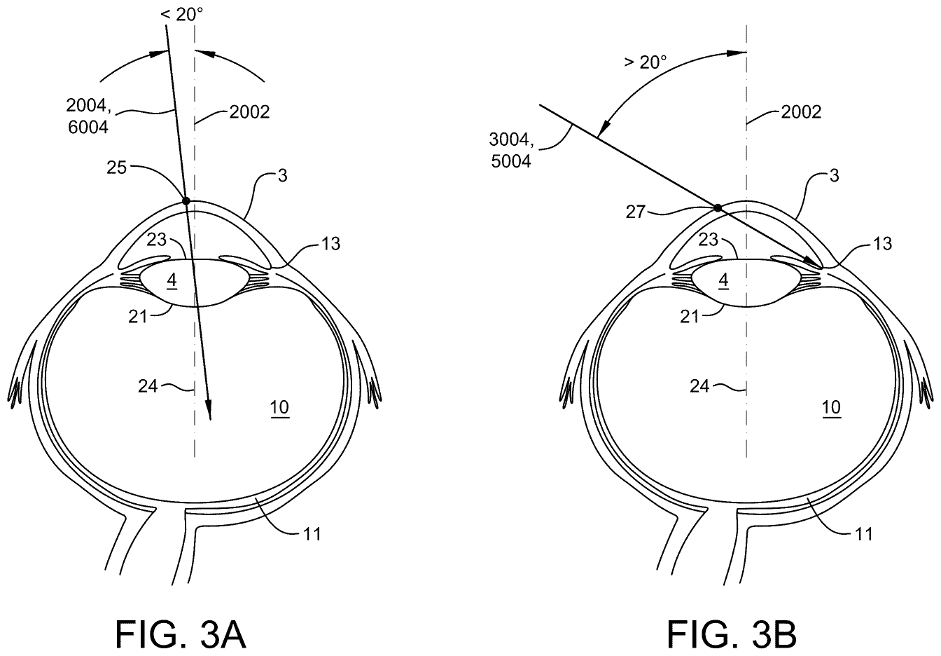

With reference to FIGS. 2, 3A, and 3B, an ophthalmic system 1000 adapted for conversion from an on-axis to an off-axis system includes an on-axis system 2000 having an on-axis-system axis 2002 and an off-axis adapter 3000 having an off-axis-adapter axis 3002. The on-axis system 2000 can be an ophthalmic surgical system designed for on-axis surgery, such as corneal and cataract surgery. The on-axis system 2000 includes a distal end 2006 and optics that are configured to provide an on-axis beam path 2004 that extends through the distal end and can be aligned with an on-axis target structure, e.g., targets in the anterior segment of the eye such as the crystalline lens 4, the posterior capsule of the lens, the anterior capsule of the lens, the vitreous humor 10, or the retina 11.

The off-axis adapter 3000 includes a distal end 3006 and optics. The off-axis adapter 3000, also referred to herein as an off-axis attachment or off-axis subsystem, is configured to couple to and decouple from the on-axis system 2000. When coupled to the on-axis system 2000, the optics of the off-axis adapter 3000 are optically aligned with the on-axis beam path 2004. The optics of the off-axis adapter 3000 are configured to provide an off-axis beam path 3004 that extends through the distal end and can be aligned with an off-axis target structure, e.g., the irido-corneal angle 13. Thus, the off-axis adapter 3000 converts the on-axis system 2000 that is only able to access the anterior segment of the eye to a system that accesses the irido-corneal angle 13.

With reference to FIGS. 2 and 3A, the on-axis system 2000 can be configured to direct a light beam along an on-axis beam path 2004 that is aligned with an on-axis target structure. In this case, the on-axis beam path 2004 is parallel or substantially parallel to the on-axis-system axis 2002 and also parallel or substantially parallel to the optical axis 24 of the eye 1, provided the on-axis-system axis 2002 and the optical axis of the eye are substantially coaxially aligned.

Substantially coaxially aligned means the on-axis-system axis 2002 and the optical axis 24 of the eye are at least within 10 degrees of being coaxial with each other and laterally offset not more than 2 mm at the distal surface of the optical element 700a. This is a practical limitation mostly driven by how accurately the eye can be attached to a patient interface 800, including the patient's ability to keep their eye steady during the attachment process. Coaxial alignment therefore varies from patient to patient and manifests as an angular misalignment and a lateral offset. The ophthalmic system 1000 is designed to tolerate variances in coaxial alignment, however excessive misalignment of the eye renders some portions of the targeted tissue inaccessible through the limited exit aperture of the ophthalmic system.

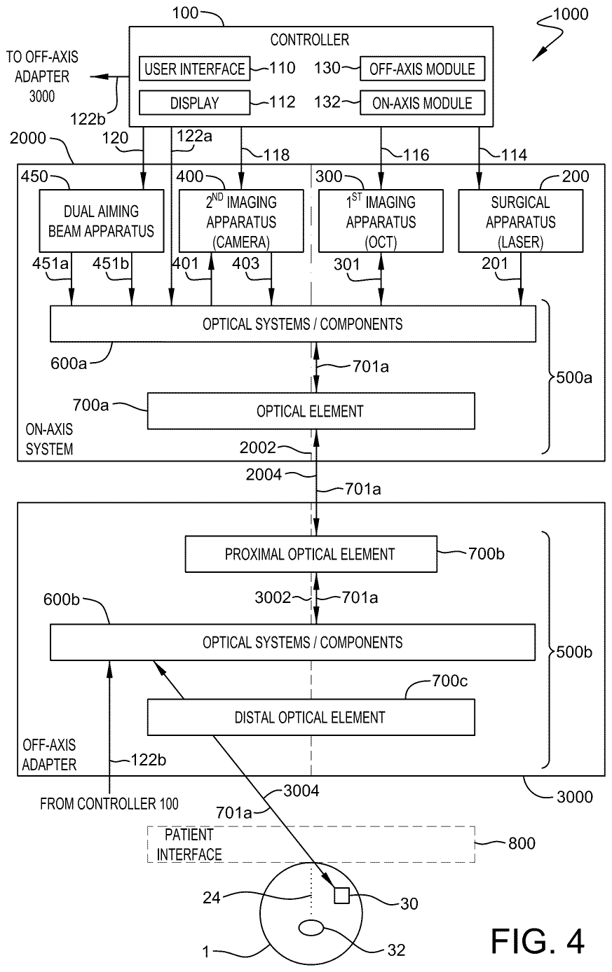

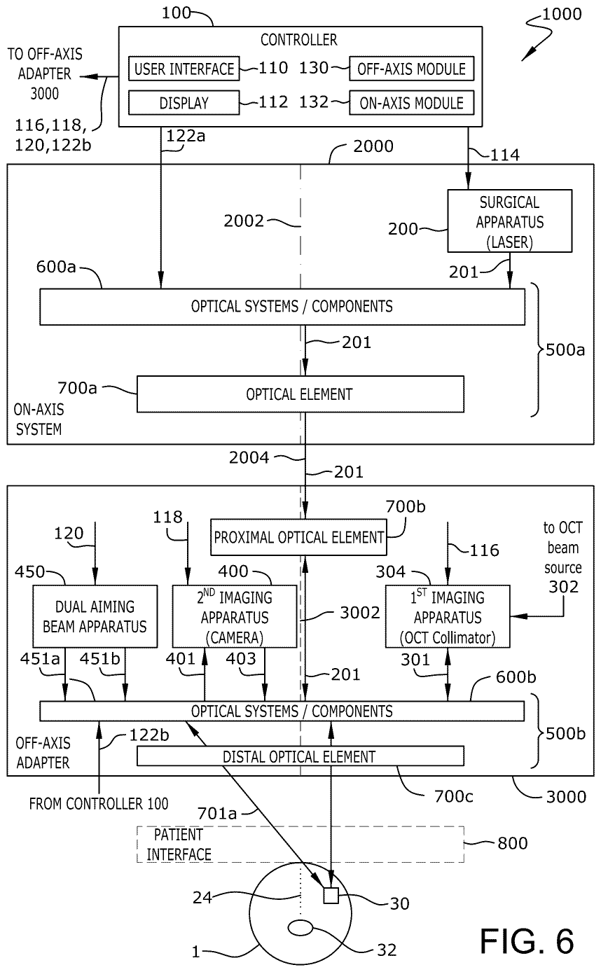

Substantially parallel means the on-axis beam path 2004 is at an angle within an on-axis threshold angle of parallel with the on-axis-system axis 2002, where the on-axis threshold angle is measured relative to the point 25 where the on-axis beam path 2004 exits the most distal optical element 700a (as shown in FIGS. 4 and 6) of the on-axis-system axis 2002, and is in the range of 0 to 20 degrees. A light beam aligned with an on-axis beam path 2004 may access an on-axis target of the eye, such as a region of the cornea 3, the crystalline lens 4, the posterior capsule 21 of the lens, the anterior capsule 23 of the lens, the vitreous humor 10, and the retina 11. For beam paths that are beyond the on-axis threshold degree, e.g., greater than 20 degrees, managing optical aberrations of an on-axis system becomes prohibitively difficult without adding excessive complexity, weight and cost to the optical system.

With reference to FIGS. 2 and 3B, the off-axis adapter 3000 disclosed herein is configured to change/alter the alignment of a light beam path provided by the on-axis system 2000 from an on-axis beam path 2004 to an off-axis beam path 3004 that is non-parallel to the on-axis-system axis 2002 and also non-parallel to the optical axis 24 of the eye 1, provided the on-axis-system axis 2002 and the optical axis of the eye are substantially coaxially aligned with an off-axis-adapter axis 3002.

Substantially coaxially aligned means the on-axis-system axis 2002, the off-axis-adapter axis 3002, and the optical axis 24 of the eye 1 are at least within 10 degrees of being coaxial with each other and laterally offset not more than 2 mm.

Non-parallel means the off-axis beam path 3004 is at an off-axis threshold angle from the on-axis-system axis 2002, where the off-axis threshold angle is measured relative to the point 27 where the off-axis beam path 3004 exits the most distal optical element 700c (as shown in FIGS. 4 and 6) of the off-axis-adapter axis 3002, and is in the range of 20 to 90 degrees. A light beam aligned along an off-axis beam path 3004 may access an off-axis target of the eye, such as a region of the irido-corneal angle 13 of the eye.

As disclosed further below, in a first configuration of an adaptable ophthalmic system 1000, the off-axis adapter 3000 only includes optics that change/alter the alignment of a light beam path provided by the on-axis system from an on-axis beam path 2004 to an off-axis beam path 3004. In a second configuration of an ophthalmic system 1000, in addition to such optics the off-axis adapter 3000 further includes imaging related components.

Regarding the term “light beam,” in the following description “light beam” or “beam” may-depending on the context-refer to one of a laser beam, an OCT beam, an illumination beam, a visual observation beam, dual aiming beams, or any other type of light beam. The term “colinear beams” refers to two or more different beams that are combined by optics to share a same optical beam path to a same target location of the eye as they enter the eye. The term “non-colinear beams” refers to two or more different light beams that have different optical beam paths into the eye. The term “co-targeted beams” refers to two or more different light beams that have different optical beam paths into the eye but that target a same location of the eye. In colinear beams, the different light beams may be combined to share a same optical beam path into the eye by dichroic or polarization beam splitters, and delivered along a same optical beam path through a multiplexed delivery of the different light beams. In non-colinear beams, the different light beams are delivered into the eye along different optical beam paths that are separated spatially or by an angle between them. In the description to follow, any of the foregoing light beams or combined beams may be generically referred to as a light beam. The terms distal and proximal may be used to designate the direction of travel of a beam, or the physical location of components relative to each other within the integrated surgical system. The distal direction refers to a direction toward the eye. The proximal direction refers to a direction away from the eye.

### First Configuration of On-Axis to Off-Axis System

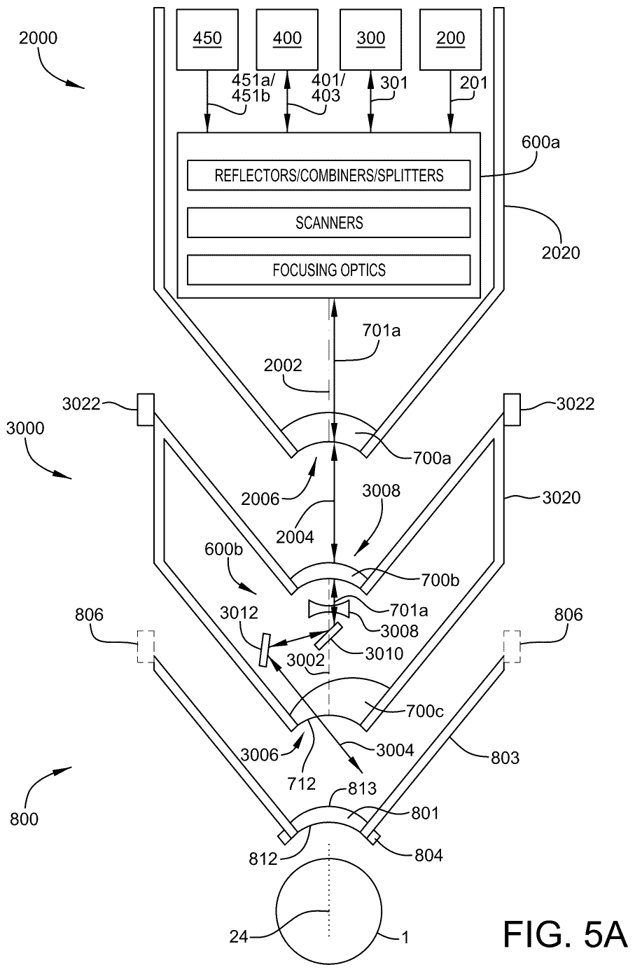

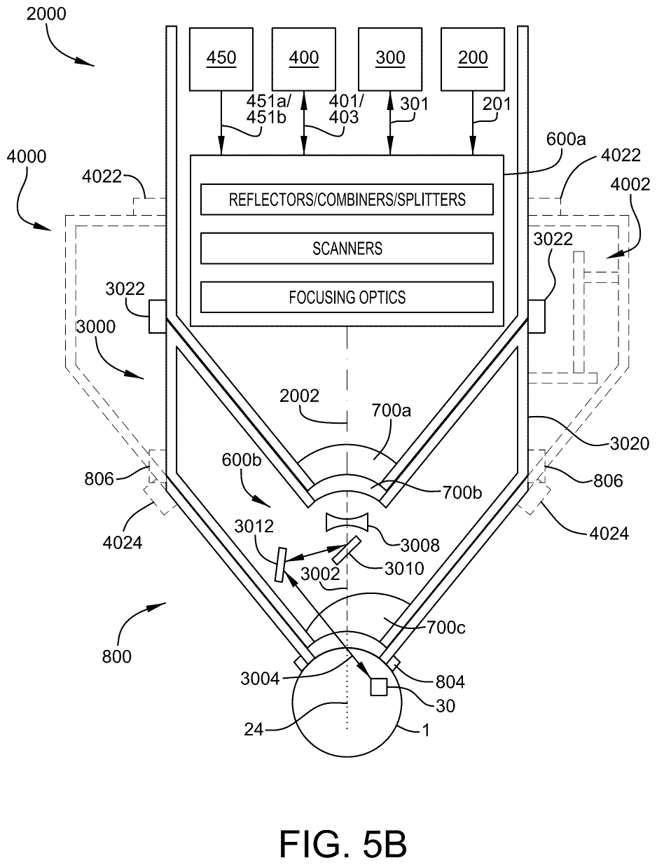

With reference to FIGS. 4, 5A, and 5B, a first configuration of an adaptable ophthalmic system 1000 includes an on-axis system 2000 and an off-axis adapter 3000 that only includes optics that functions to alter or adapt or change an on-axis beam path 2004 of the on-axis system 2000 to an off-axis beam path 3004. The off-axis adapter 3000 is configured to couple to and decouple from the on-axis system 2000.

In some embodiments, the off-axis adapter 3000 is configured to directly couple with the on-axis system 2000. To this end, and with reference to FIGS. 5A and 5B, the off-axis adapter 3000 includes an attachment mechanism 3022 that enables the coupling and decoupling of the off-axis adapter 3000 from the on-axis system 2000. The attachment mechanism 3022 may be one of a bayonet mount, clamp, vacuum attachment, electro-magnetic attachment, which can be operated by hand or with an automated mechanism, without using any tools. When directly coupled together, the on-axis system 2000 and the off-axis adapter 3000 are optically aligned such that a light beam exiting the on-axis system 2000 enters the off-axis adapter 3000, and likewise, a light beam exiting the off-axis adapter 3000 enters the on-axis system 2000.

In some embodiments, the off-axis adapter 3000 is configured to be indirectly coupled with the on-axis system 2000. To this end, and with reference to FIG. 5B, a rotation attachment 4000 is configured to directly couple and decouple from a patient interface 800 at one end and the on-axis system 2000 at the other end, with the off-axis adapter 3000 held in place between the two. A proximal attachment mechanism 4022 enables coupling and decoupling with the on-axis system 2000, while a distal attachment mechanism 4024 enables coupling and decoupling with the patient interface 800. The attachment mechanisms 4022, 4024 may be one of a bayonet mount, clamp, vacuum attachment, electro-magnetic attachment, which can be operated by hand or with an automated mechanism, without using any tools. As disclosed later below, the rotation attachment 4000 is configured to rotate the off-axis adapter 3000 or components thereof relative to the patient interface 800 and the on-axis system 2000. When indirectly coupled together, the on-axis system 2000 and the off-axis adapter 3000 are optically aligned such that a light beam exiting the on-axis system 2000 enters the off-axis adapter 3000, and likewise, a light beam exiting the off-axis adapter 3000 enters the on-axis system 2000.

The on-axis system 2000 includes one or more apparatuses 200, 300, 400, 450 that output and/or receive light beams, and optics 500a arranged and configured to direct light beams along an on-axis beam path 2004 that is within an on-axis threshold angle of an on-axis-system axis 2002 of the on-axis system, where the on-axis threshold angle is in the range of 0 to 20 degrees. The apparatuses of the on-axis system 2000 can include a surgical apparatus 200, a first imaging apparatus 300, a second imaging apparatus 400, and a dual aiming beam apparatus 450. The optics 500a of the on-axis system 2000 includes optical systems/components 600a and an optical element 700a at the distal end 2006 of a housing 2020 of the on-axis system 2000. The on-axis-system axis 2002 is coincident with the mechanical center of the optical element 700a.

The surgical apparatus 200 may be a femtosecond laser source that outputs a laser beam 201. The first imaging apparatus may be an OCT imaging apparatus 300 that outputs an OCT beam 301. The second imaging apparatus may be a camera apparatus 400 that provides illumination 403 and captures a visual observation beam 401. The camera may be a digital camera fitted with a goniolens to provide gonioscopic images of the eye. The illumination 403 may be provided by LEDs or light delivered via fiber optic cables. The dual aiming beam apparatus 450 that outputs a pair of beams of light, referred to herein as dual aiming beams 451a/451b, for use in detecting a surface of ocular tissue. A dual aiming beam apparatus 450 is disclosed in U.S. Pat. No. 11,564,567, title “System and Method for Locating a surface of Ocular Tissue for Glaucoma Surgery Based on Dual Aiming Beams,” the contents of which are incorporated herein by reference.

The optical systems/components 600a of the on-axis system 2000 may include reflectors, beam combiners, and beam splitters. These components may include dichroic or polarization beam splitters that split and recombine light beams with different wavelength and/or polarization. These components may also include optics to change certain parameters of the individual light beams such as beam size, beam angle and divergence. Two or more of the laser beam 201, the OCT beam 301, the visual observation beam 401, and the dual aiming beams 451a/451b may be combined with dichroic, polarization or other kind of beam combiners and provided to the optical element 700a as a combined beam. For example, the OCT beam 301 can be colinearly combined with the laser beam 201. Likewise, the dual aiming beams 451a/451b can be colinearly combined with the laser beam 201. The path of the visual observation beam 401 may be coaxial with the path of the laser beam 201. Going forward, a light beam identified by reference number 701a may be an individual beam such as a laser beam 201, an OCT beam 301, a visual observation beam 401, dual aiming beams 451a/451b or any other type of light beam, or a combination of two or more of these individual beams.

With continued reference to FIGS. 4, 5A, and 5B, in some embodiments the optical systems/components 600a of the on-axis system 2000 include separate scanners that scan the laser beam 201 and OCT beam 301 independent of each other. In some embodiments, the optical systems/components 600a of the on-axis system 2000 include a single scanner that scans the laser beam 201 and OCT beam 301. For scanning transversal to a light beam 201, 301, angular scanning galvanometer scanners may be used. The optical systems/components 600a of the on-axis system 2000 may include focusing optics, e.g., focus lenses and linear stages, for affecting the focus of a laser beam 201, an OCT beam 301, the camera apparatus 400, and the dual aiming beams 451a/451b.

The optical element 700a of the on-axis system 2000 is optically coupled to receive one or more light beams 701a from the optical systems/components 600a. The optical element 700a is configured to output the one or more light beams 701a in the proximal direction along the on-axis beam path 2004. With respect to light beams 701a travelling in the proximal direction, such as the visual observation beam 401, the optical element 700a receives the light beam from the off-axis adapter 3000 along the on-axis beam path 2004 and outputs it to the optical system/components 600a.

The off-axis adapter 3000 includes optics 500b arranged and configured to direct light beams along an off-axis beam path 3004 that is greater than an off-axis threshold angle from an off-axis-adapter axis 3002, where the off-axis threshold angle is in the range of 20 to 90 degrees. The optics 500b of the off-axis adapter 3000 includes a proximal optical element 700b and a distal optical element 700c. The proximal optical element 700b is at the proximal end 3007 of a housing 3020 of the off-axis adapter 3000 and is configured to optically couple with the optical element 700a of the on-axis system 2000. The distal optical element 700c is at the distal end 3006 of a housing 3020 and is configured to optically couple with an optional patient interface 800. The off-axis-adapter axis 3002 is coincident with the mechanical centers of the proximal optical element 700b and the distal optical element 700c. The optics 500b of the off-axis adapter 3000 also includes optical systems/components 600b between the proximal optical element 700b and the distal optical element 700c.

The ophthalmic system 1000 may include a patient interface 800. The patient interface 800 immobilizes the eye relative to components of the ophthalmic system 1000, creates a sterile barrier between the components and the patient, and provides optical access between the eye and the components. The patient interface 800 includes an optical window 801. The optical window 801 is surrounded by a wall 803 of the patient interface 800 and an immobilization device, such as a suction ring 804. When the suction ring 804 is in contact with the eye 1, an annular cavity is formed between the suction ring and the eye. When vacuum is applied to the suction ring 804 and the cavity via a vacuum tube a vacuum pump (not shown), vacuum forces between the eye and the suction ring attach the eye to the patient interface 800. Removing the vacuum releases or detaches the patient interface 800 from the eye 1. In some embodiments, the end of the patient interface 800 opposite the eye 1 includes an attachment interface 806 configured to attach to the housing 3020 of the off-axis adapter 3000 to thereby affix the position of the eye relative to the other components of the ophthalmic system 1000. The attachment interface 806 can work with mechanical, vacuum, magnetic or other principles and is detachable from the housing 3020. In cases where a rotation attachment 4000 is present and rotation of the off-axis adapter 3000 is desired, the attachment interface 806 of the patient interface is not activated, or alternatively, an embodiment of a patient interface that does not include the attachment interface 806 is used.

The optical window 801 has a concave surface 812 and a convex surface 813 opposite the concave surface. The concave surface 812 is configured to couple to the eye, either through a direct contact or through index matching material, liquid, or gel, placed in between the concave surface 812 and the eye 1. The shape of the convex surface 813 matches the shape of the distal surface 712 of the distal optical element 700c to enable rotation of the off-axis adapter 3000 inside the patient interface 800 without any rotational torque being transferred to the patient interface that is secured to the eye. Likewise, facing surfaces of a housing 3020 of the off-axis adapter 3000 and the wall 803 of the patient interface 800 are complementary shaped so as to physically mate together. Furthermore, the facing surfaces of a housing 3020 of the off-axis adapter 3000 and the wall 803 of the patient interface 800 are configured to enable rotation of the off-axis adapter without any rotational torque being transferred to the patient interface. To this end, the respective surfaces may be formed of a material having a low coefficient of friction. Optical coupling liquids, such as water or oil or coupling gels may be applied between the mating surfaces to facilitate light transmission and to further reduce friction.

With reference to FIGS. 5A and 5B, the optical element 700a of the on-axis system 2000 and the proximal optical element 700b of the off-axis adapter 3000 are configured to optically couple together such that the on-axis-system axis 2002 and the off-axis-adapter axis 3002 are substantially coaxially aligned. To this end, facing surfaces of the optical element 700a of the on-axis system 2000, and the proximal optical element 700b of the off-axis adapter 3000 are complementary shaped, e.g., one is concave while the other is convex, so as to physically mate together. Likewise, facing surfaces of a housing 2020 of the on-axis system 2000 and a housing 3020 of the off-axis adapter 3000 are complementary shaped so as to physically mate together. Furthermore, the facing surfaces of a housing 2020 of the on-axis system 2000 and a housing 3020 of the off-axis adapter 3000 are configured to enable rotation of the off-axis adapter without any rotational torque being transferred to the on-axis system. To this end, the respective surfaces may be formed of a material having a low coefficient of friction. Optical coupling liquids, such as water or oil or coupling gels may be applied between the mating surfaces to facilitate light transmission and to further reduce friction.

With continued reference to FIGS. 5A and 5B, the distal optical element 700c of the off-axis adapter 3000 and the optical window 801 of the patient interface 800 are configured to optically couple together such that the off-axis-adapter axis 3002 and the optical axis 24 of the eye 1 are substantially coaxially axially aligned. To this end, facing surfaces of the distal optical element 700c of the off-axis adapter 3000 and the optical window 801 of the patient interface 800 are complementary shaped, e.g., one is concave while the other is convex, so as to physically mate together. Likewise, facing surfaces of the housing 3020 of the off-axis adapter 3000 and the wall 803 of the patient interface 800 are complementary shaped so as to physically mate together. The facing surface, i.e., the concave surface 812, of the distal optical element 700c is also configured for direct placement on the eye 1. Thus, in the absence of patient interface 800, the off-axis adapter 3000 can couple directly with the eye 1.

As shown in FIG. 5B, when coupled to the patient interface 800, the off-axis adapter 3000 is placed relative to the eye 1 such that the off-axis-adapter axis 3002 is substantially coaxially aligned with the optical axis 24 of the eye. The patient interface 800 maintains this alignment and prevents sliding of the distal optical element 700c relative to the eye 1.

The optical systems/components 600b of the off-axis adapter 3000 may include reflectors, e.g., a pair of mirrors 3010, 3012, arranged to receive and direct light beams between the on-axis beam path 2004 and the off-axis beam path 3004. More specifically, the pair of mirrors 3010, 3012 is arranged to receive one or more light beams 701a from the on-axis system 2000 along the on-axis beam path 2004 and to redirect the one or more light beams to the off-axis beam path 3004. Likewise, the pair of mirrors 3010, 3012 is arranged to receive one or more light beams 701a along the off-axis beam path 3004 and to redirect the one or more light beams to the on-axis beam path 2004.

The optical systems/components 600b of the off-axis adapter 3000 may include focusing optics 3008, e.g., focus lenses and linear stages, for affecting the focus of a laser beam 201, an OCT beam 310, the camera apparatus 400, and the dual aiming beams 451a/451b. More specifically, the focusing optics 3008 can include a focus extender optical assembly configured to convert/extend the focal range of a light beam 701a as established by optics 500a of the on-axis system 2000 to reach the off-axis target 30. The focusing optics 3008 can also be configured to adjust light beam characteristic. For example, in the case of a laser beam 201, the focusing optics 3008 can adjust laser beam parameters, beam divergence and beam size to ensure the laser is focused with the correct numerical aperture to produce the desired spot size at the off-axis target 30.

It is understood by those skilled in the art that adding or removing planar beam folding mirrors or other types of reflecting surfaces or other focusing optics does not alter the principal working of the optical systems/components 600b. It is also understood that the configuration of optical systems/components 600b shown in FIGS. 4, 5A and 5B are schematic in nature and that numerous other configurations and arrangements are possible.

With reference to FIG. 4, the ophthalmic system 1000 also includes a controller 100. The controller 100 of the ophthalmic system 1000 includes a user interface 110 and a display 112. The user interface 110 accepts commands from a user that initiates one or more of delivery of laser therapy by the laser apparatus 200, imaging by the OCT imaging apparatus 300, imaging by the camera apparatus 400, and surface locating by the dual the dual aiming beam apparatus 450. The display 112 displays images generated by the OCT imaging apparatus 300 and images captured by the camera apparatus 400.

Control signals 114 from the controller 100 to laser apparatus 200 function to control internal and external operation parameters of the laser source, including for example, power, repetition rate and beam shutter. Control signals 116 from the controller 100 to the OCT imaging apparatus 300 function to control OCT beam parameters, and the acquiring, analyzing, and displaying of OCT images. Control signals 118 from the controller 100 to the camera apparatus 400 function to control the capturing, image processing and displaying of video images. Control signals 120 from the controller 100 to the dual aiming beam apparatus 450 function to control the output of beams of light by the one or more aiming beam sources of the dual aiming beam apparatus.

Control signals 122a from the controller 100 to optical systems/components 600a of the on-axis system 2000 function to control the scanning of the laser beam 201 and scanning of the OCT beam 301, and to control the focus of the laser beam 201, the focus of the OCT beam 301, the focus of the camera apparatus 400, and the focus of the dual aiming beams 451a/451b. Control signals 122b from the controller 100 to the optical systems/components 600b of the off-axis adapter 3000 also function to control the focus of the laser beam 201, the focus of the OCT beam 301, the focus of the camera apparatus 400, and the focus of the dual aiming beams 451a/451b.

The controller 100 can include an off-axis module 130, e.g., software instructions, that controls the surgical apparatus 200 and optics 500a to deliver treatment to off-axis targets 30, and an on-axis module 132 that controls the surgical apparatus 200 and optics 500a to deliver treatment to on-axis targets 32. The respective modules 130, 132 can reconfigure the surgical apparatus 200, e.g., laser engine, to a set of parameters, such as laser energy and repetition rate that are appropriate for the target 30, 32, and the optics 500a, e.g., scanners, to a set of parameters, such as the x-y-z scanning dimensions, the scanning speed, and the scan pattern, that are appropriate for the target 30, 32. The controller 100 is configured so that the off-axis module 130 controls when the off-axis adapter 3000 is present, e.g. coupled to the on-axis system 2000; and the on-axis module controls when the off-axis adapter 3000 is not present.

With reference to FIG. 5B, as disclosed above, in some embodiments the off-axis adapter 3000 is coupled to the on-axis system 2000 by a rotation attachment 4000 that attaches to the patient interface 800 at one end and the on-axis system 2000 at the other end, and enables rotation of the off-axis adapter. The rotation attachment 4000 includes a rotation mechanism 4002 configured to rotate the off-axis adapter 3000 relative to the patient interface 800 and the on-axis system 2000. The rotation mechanism 4002 rotates the off-axis adapter 3000 around the off-axis-adapter axis 3002 without imparting any rotation to the patient interface 800 or the on-axis system 2000. In other words, the off-axis adapter 3000 rotates but the patient interface 800 and the on-axis system 2000 do not. This enables the ophthalmic system 1000 to access different segments of a region of the eye 1, such as different circumferential portions of the irido-corneal angle 13, without having to decouple the off-axis adapter 3000 from the on-axis system 2000 and the patient interface 800. In some embodiments, the rotation mechanism 4002 enables manual rotation of the off-axis adapter 3000. For example, the rotation mechanism 4002 can be a geared thumbwheel that engages a surface of the housing 3020 of the off-axis adapter 3000, where rotation of the thumbwheel causes the off-axis adapter to rotate. In some embodiments, the rotation mechanism 4002 is a motorized gear that engages the outer wall of the housing 3020 of the off-axis adapter 3000. In this case, rotation of the off-axis adapter 3000 can occur upon user activation through the user interface 110 and the rotation is controlled by the controller 100. In some embodiments, the rotation attachment 4000 may be configured to rotate components of the off-axis adapter 3000 relative to the patient interface 800 and the on-axis system 2000 without rotating the housing 3020 of the off-axis adapter. In this case, the rotation mechanism 4002 may extend through the housing 3020 of the off-axis adapter to mechanically engage an assembly of one or more components, e.g., the pair of mirrors 3010, 3012 of the optical systems/components 600b, for rotation.

### Second Configuration of On-Axis To Off-Axis System

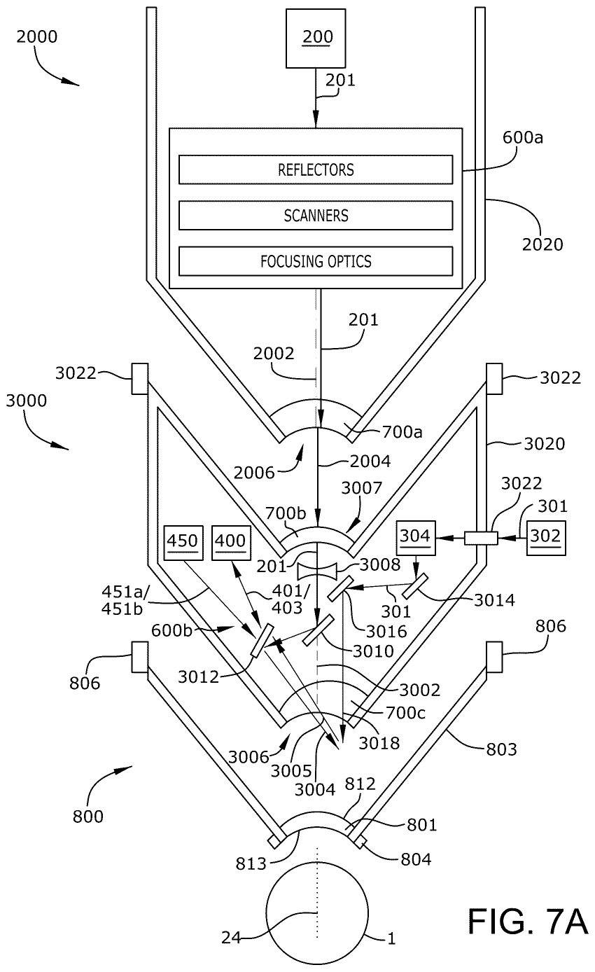

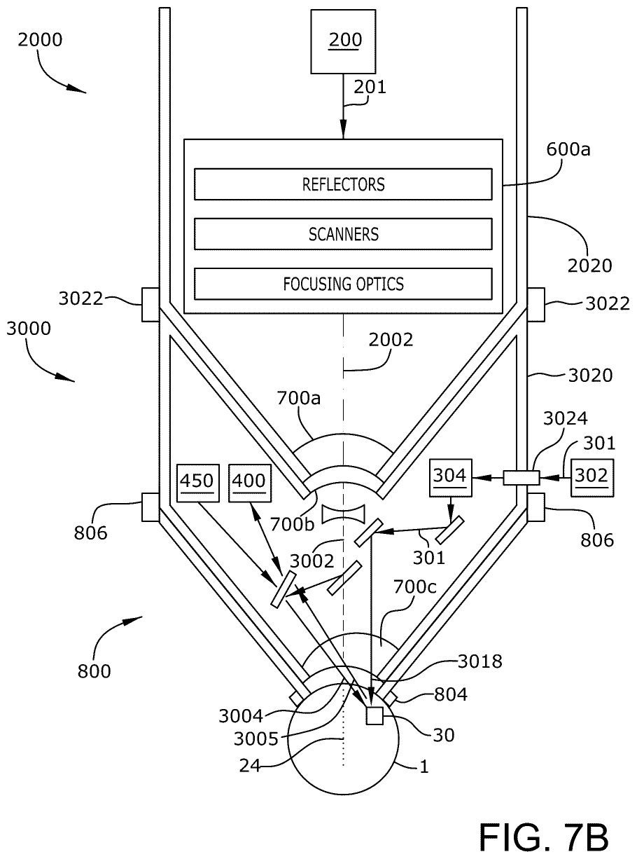

With reference to FIGS. 6, 7A, and 7B, a second configuration of an ophthalmic system 1000 includes an on-axis system 2000 that includes a surgical apparatus, and an off-axis adapter 3000 that includes one or more imaging apparatuses and optics that functions to alter or adapt or change an on-axis beam path 2004 of the on-axis system 2000 to an off-axis beam path 3004. The off-axis adapter 3000 is configured to couple to and decouple from the on-axis system 2000.

In some embodiments the off-axis adapter 3000 is configured to directly couple with the on-axis system 2000. To this end, and with reference to FIGS. 7A and 7B, the off-axis adapter 3000 includes an attachment mechanism 3022 that enables the coupling and decoupling of the off-axis adapter 3000 from the on-axis system 2000. The attachment mechanism 3022 may be one of a bayonet mount, clamp, vacuum attachment, electro-magnetic attachment, which can be operated by hand or with an automated mechanism, without using any tools. When directly coupled together, the on-axis system 2000 and the off-axis adapter 3000 are optically aligned such that a light beam exiting the on-axis system 2000 enters the off-axis adapter 3000, and likewise, a light beam exiting the off-axis adapter 3000 enters the on-axis system 2000.

The on-axis system 2000 includes a surgical apparatus 200 that outputs a surgical light beam and optics 500a arranged and configured to direct the surgical light beam along an on-axis beam path 2004 that is within an on-axis threshold angle of an on-axis-system axis 2002 of the on-axis system, where the on-axis threshold angle is in the range of 0 to 20 degrees. The surgical apparatus of the on-axis system 2000 can be a femtosecond laser source that outputs a laser beam 201. The optics 500a of the on-axis system 2000 includes optical systems/components 600a and an optical element 700a at the distal end 2006 of a housing 2020 of the on-axis system 2000. The on-axis-system axis 2002 is coincident with the mechanical center of the optical element 700a.

The optical systems/components 600a of the on-axis system 2000 may include reflectors arranged to receive the laser beam 201 and to direct the laser beam into alignment with the on-axis beam path 2004 of the on-axis system 2000. The optical systems/components 600a of the on-axis system 2000 may include a scanner for scanning the laser beam 201. For scanning transversal to a laser beam 201 an angular scanning galvanometer scanner may be used. The optical systems/components 600a of the on-axis system 2000 may include focusing optics, e.g., focus lenses and linear stages, for affecting the focus of a laser beam 201. These components may also include optics to change certain parameters of the individual light beams such as beam size, beam angle and divergence.

The optical element 700a of the on-axis system 2000 is optically coupled to receive the laser beam 201 from the optical systems/components 600a. The optical element 700a is configured to output the laser beam 201 in the proximal direction along the on-axis beam path 2004.

In some embodiments, the off-axis adapter 3000 includes one or more imaging apparatuses that output and/or receive light beams and optics 500b arranged and configured to direct light beams along an off-axis beam path 3004 that is greater than an off-axis threshold angle from an off-axis-adapter axis 3002, where the off-axis threshold angle is in the range of 20 to 90 degrees. The imaging apparatuses of the off-axis adapter 3000 may include a component 304 of a first imaging apparatus, a second imaging apparatus 400, and a dual aiming beam apparatus 450. The component of the first imaging apparatus may be an OCT beam collimator 304 that receives an OCT beam 301 from a OCT beam source 302 through a coupling 3024 of a housing 3020 of the off-axis adapter 3000. The second imaging apparatus may be a camera apparatus 400 that provides illumination 403 and captures a visual observation beam 401. The OCT beam 301 is preferably delivered to the coupling 3024 via an optical fiber, the flexibility of the fiber allowing free rotation of the off-axis adapter 3000. In this case the coupling 3024 is a fiber optic connector.

The optics 500b of the off-axis adapter 3000 include a proximal optical element 700b and a distal optical element 700c. The proximal optical element 700b is at the proximal end 3007 of the housing 3020 of the off-axis adapter 3000 and is configured to optically couple with the optical element 700a of the on-axis system 2000. The distal optical element 700c is at the distal end 3006 of a housing 3020 and is configured to optically couple with an optional patient interface 800. The off-axis-adapter axis 3002 is coincident with the mechanical centers of the proximal optical element 700b and the distal optical element 700c.

The optics 500b of the off-axis adapter 3000 also include optical systems/components 600b between the proximal optical element 700b and the distal optical element 700c.

The ophthalmic system 1000 may include a patient interface 800 like the one describe above with reference to FIGS. 5A and 5B. When coupled to the patient interface 800, the off-axis adapter 3000 is placed relative to the eye 1 such that the off-axis-adapter axis 3002 is substantially coaxially aligned with the optical axis 24 of the eye. The patient interface 800 maintains this alignment and prevents sliding of the distal optical element 700c relative to the eye 1.

The optical systems/components 600b may include reflectors, beam combiners, and beam splitters. These components may include dichroic or polarization beam splitters that split and recombine light beams with different wavelength and/or polarization. These components may also include optics to change certain parameters of the individual light beams such as beam size, beam angle and divergence. In the example configuration shown in FIG. 7A, the optical systems/components 600b includes reflectors, e.g., a pair of mirrors 3010, 3012, arranged to receive the laser beam 201 from the on-axis system 2000 along the on-axis beam path 2004 and to redirect the light beam to the off-axis beam path 3004.

In the example configuration shown in FIG. 7A, light beams traveling in the distal direction, e.g., the dual aiming beams 451a/451b and the laser beam 201, are input to a dichroic mirror 3012. The dichroic mirror 3012 combines the dual aiming beams 451a/451b with the laser beam 201 and provides the combined beam to the distal optical element 700c along a first off-axis beam path 3004 that exits the distal optical element at an angle greater than an off-axis threshold angle from the on-axis-system axis 2002. Light beams traveling in the proximal direction, e.g., the visual observation beam 401, are received by the dichroic mirror 3012 from the distal optical element 700c along a second off-axis beam path 3005 at an angle greater than the off-axis threshold angle from the on-axis-system axis 2002. The dichroic mirror 3012, maintains separation between the visual observation beam 401 and the dual aiming beams 451a/451b and the laser beam 201. In the example configuration shown in FIG. 7A, the optical systems/components 600b includes reflectors, e.g., a pair of mirrors 3014, 3016, arranged to receive the OCT beam 301 from the OCT beam collimator 304 and to redirect the OCT beam to a beam path 3018 radially offset from the on-axis-system axis 2002 and radially offset from the off-axis-adapter axis 3002.

The optical systems/components 600b of the off-axis adapter 3000 may include a scanner that scans the OCT beam 301. In the example configuration shown in FIG. 7A, the mirror 3016 is also configured to scan the OCT beam 301. In this case, the mirror 3016 may be an angular scanning galvanometer scanner or a 2D MEMS/controllable mirror system. For scanning transversal to the OCT beam 301 an angular scanning galvanometer scanner may be used.

The optical systems/components 600b of the off-axis adapter 3000 may include focusing optics 3008, e.g., focus lenses and linear stages, for affecting the focus of a laser beam 201. More specifically, the focusing optics 3008 can be a focus extender configured to convert/extend the focal range of the laser beam 201 to reach the off-axis target 30. The focusing optics 3008 can also adjust laser beam characteristic. For example, the focusing optics 3008 can adjust laser beam parameters, beam divergence and beam size to ensure the laser is focused with the correct numerical aperture to produce the desired spot size at the off-axis target 30.

It is understood by those skilled in the art that adding or removing planar beam folding mirrors or other types of reflecting surfaces or other focusing optics does not alter the principal working of the optical systems/components 600b. It is also understood that the configuration of optical systems/components 600b shown in FIGS. 6, 7A and 7B are schematic in nature and that numerous other configurations and arrangements are possible.

With continued reference to FIG. 6, the ophthalmic system 1000 also includes a controller 100. The controller 100 of the ophthalmic system 1000 includes a user interface 110 and a display 112. The user interface 110 accepts commands from a user that initiates one or more of delivery of laser therapy by the laser apparatus 200, imaging by the OCT imaging apparatus 300, imaging by the camera apparatus 400, and surface locating by the dual the dual aiming beam apparatus 450. The display 112 displays images generated by the OCT imaging apparatus 300 and images captured by the camera apparatus 400.

Control signals 114 from the controller 100 to laser apparatus 200 function to control internal and external operation parameters of a laser source, including for example, power, repetition rate and beam shutter. Control signals 116 from the controller 100 to the OCT beam collimator 304 function to control OCT beam parameters, and the acquiring, analyzing, and displaying of OCT images. Control signals 118 from the controller 100 to the camera apparatus 400 function to control the capturing, image processing and displaying of video images. Control signals 120 from the controller 100 to the dual aiming beam apparatus 450 function to control the output of beams of light by the one or more aiming beam sources of the dual aiming beam apparatus.

Control signals 122a from the controller 100 to optical systems/components 600a of the on-axis system 2000 function to control the scanning of the laser beam 201 and the focus of the laser beam 201. Control signals 122b from the controller 100 to the optical systems/components 600b of the off-axis adapter 3000 function to further control the focus of the laser beam 201, and to control the focus of the OCT beam 301, the focus of the camera apparatus 400, and the focus of the dual aiming beams 451a/451b.

The controller 100 can include an off-axis module 130, e.g., software instructions, that controls the surgical apparatus 200 and optics 500a to deliver treatment to off-axis targets 30, and an on-axis module 132 that controls the surgical apparatus 200 and optics 500a to deliver treatment to on-axis targets 32. The respective modules 130, 132 can reconfigure the surgical apparatus 200, e.g., laser engine, to a set of parameters, such as laser energy and repetition rate that are appropriate for the target 30, 32, and the optics 500a, e.g., scanners, to a set of parameters, such as the x-y-z scanning dimensions, the scanning speed, and the scan pattern, that are appropriate for the target 30, 32. The controller 100 is configured so that the off-axis module 130 controls when the off-axis adapter 3000 is present, e.g. coupled to the on-axis system 2000; and the on-axis module controls when the off-axis adapter 3000 is not present.

### Method of Accessing Tissue Targets

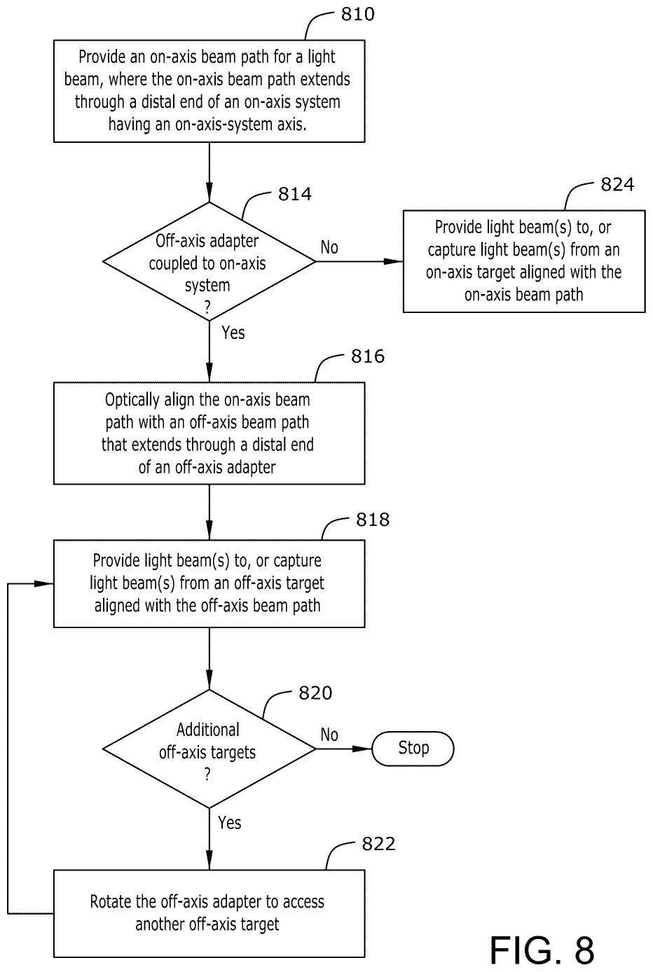

With reference to FIG. 8, a method of enabling access to either on-axis or off-axis targets in the eye is described. The method may be performed using the adaptable ophthalmic system 1000 described above with reference to FIGS. 4, 5A, and 5B, or the adaptable ophthalmic system 1000 described above with reference to FIGS. 6, 7A, and 7B.

At block 810, an on-axis beam path 2004 for a light beam is provided, where the on-axis beam path extends through a distal end 2006 of an on-axis system 2000. In this regard, optics 500a of the on-axis system 2000 are arranged and configured such that a distally-directed light beam, e.g., a laser beam 201 or an OCT beam 301, exiting the on-axis system through the optical element 700a exits along or in alignment with the on-axis beam path 2004. Similarly, optics 500a of the on-axis system 2000 are arranged and configured such that a proximally-directed light beam, e.g., a visual observation beam 401, entering the on-axis system through the optical element 700a is directed along or in alignment with the on-axis beam path 2004.

The on-axis system 2000 has an on-axis-system axis 2002 coincident with the mechanical center of the optical element 700a, and the on-axis beam path 2004 is within an on-axis threshold angle of the on-axis-system axis, where the on-axis threshold angle is in the range of 0 to 20 degrees. With reference to FIGS. 4, 5A, and 5B, the light beam 701a can be a combined beam comprising at least two of a laser beam 201, an OCT beam 301, a visual observation beam 401, and a pair of dual aiming beams 451a, 451b. With reference to FIGS. 6, 7A, and 7B, the light beam can be a laser beam 201.

At blocks 814/816, when an off-axis adapter 3000 is coupled to the on-axis system 2000, the on-axis beam path 2004 is diverted by redirecting optics, e.g., a pair of mirrors 3010, 3012, and optically aligned with an off-axis beam path 3004 that extends through a distal end 3006 of an off-axis adapter 3000. In this regard, optics 500b of the off-axis adapter 3000 are arranged and configured such that a distally directed light beam, e.g., a laser beam 201 or an OCT beam 301, exiting the off-axis adapter 3000 through the distal optical element 700c exits along or in alignment with the off-axis beam path 3004. Similarly, optics 500b of the off-axis adapter 3000 are arranged and configured such that a proximally-directed light beam, e.g., a visual observation beam 401, entering the off-axis adapter 3000 through the distal optical element 700c is directed along or in alignment with the off-axis beam path 3004.

The off-axis beam path 3004 is greater than an off-axis threshold angle from the on-axis-system axis 2002, where the off-axis threshold angle is in the range of 20 to 90 degrees. Thus, the beam path for the light beam 201, 701a is transferred from the on-axis beam path 2004 to the off-axis beam path 3004. With reference to the ophthalmic system 1000 of FIGS. 6, 7A, and 7B, the laser beam 201 can be combined with one or more of a visual observation beam 401 and a pair of dual aiming beams 451a, 451b to form a combined beam 701a that travels along the off-axis beam path 3004. In the ophthalmic system 1000 of FIGS. 6, 7A, and 7B, an OCT beam 301 is directed along a beam path independent of the off-axis beam path 3004. In other embodiments, an OCT beam can be included in the combined beam 701a.

Regarding the optical alignment of the on-axis beam path 2004 with the off-axis beam path 3004, the off-axis adapter 3000 has an off-axis-adapter axis 3002 and optics 500b, and the on-axis beam path 2004 is optically aligned with the off-axis beam path 3004 by optically coupling the on-axis beam path 2004 with the optics 500b of the off-axis adapter. To this end, the off-axis adapter 3000 is coupled with the on-axis system 2000 such that the on-axis-system axis 2002 coincident with the mechanical center of the optical element 700a is substantially coaxially aligned with the an off-axis-adapter axis 3002 that is coincident with the mechanical center of the proximal optical element 700b and the mechanical center of the distal optical element 700c. The off-axis adapter 3000 is configured to couple to and decouple from the on-axis system 2000.

At block 818, one or more apparatuses and optics of the ophthalmic system 1000 are operated to provide one or more light beams, e.g., a laser beam 201 and/or an OCT beam 301, to an off-axis target 30 or to capture one or more light beams, e.g., a visual observation beam 401, from the off-axis target. To this end, a surgical apparatus 200 may output a laser beam 201 based on a set of therapy parameters designed to treat off-axis targets 30, while one or more scanners in the optics 500a, 500b may scan the laser beam 201 and/or OCT beam 301 based on a set of scanning parameters designed to image and/or treat off-axis targets 30. In some embodiments, focusing optics of the optical systems/components 600b of the off-axis adapter 3000 are controlled to change the focus of the light beam provided by focusing optics in the optical systems/components 600a of the on-axis system 2000. For example, the focusing optics of the optical systems/components 600b of the off-axis adapter 3000 can extend the focus of the focusing optics in the optical systems/components 600a of the on-axis system 2000.

Regarding the off-axis target 30, in one method, prior to surgery, a separate gonioscopic imaging device or a combined gonioscopic/OCT imaging device is used to identify an off-axis target 30 for the surgery in the irido-corneal angle 13, and the located of the identified off-axis target 30 is noted as a circumferential angle expressed in clock-hour or in degrees of angle from a reference location. Then the orientation of the off-axis adapter 3000 is set by hand or by a motor prior to docking the patient interface 800 on the patient's eye 1.

At block 820/822, when there are additional off-axis targets 30 of interest and rotation of the off-axis adapter 3000 is enabled by a rotation attachment 4000, the off-axis adapter 3000 or components thereof can be rotated without rotating the on-axis system 2000. The rotation of the off-axis adapter 3000 or components thereof is about the off-axis-adapter axis 3002. The off-axis adapter 3000 or components thereof is rotated to align the off-axis beam path 3004 with another off-axis target 30. For example, each of an off-axis target 30 may correspond to a different portion around the circumference of the irido-corneal angle 13 of an eye 1. The process then returns to block 818. Blocks 818, 820, 822 are repeated until there are no longer any additional off-axis targets 30 of interest, at which point the process stops.

Returning to block 814, when an off-axis adapter 3000 is not coupled to the on-axis system 2000, the process proceeds to block 824, where one or more apparatuses and optics of the ophthalmic system 1000 are operated to provide one or more light beams, e.g., a laser beam 201 and/or an OCT beam 301, to an on-axis target 32 or to capture one or more light beams, e.g., a visual observation beam 401, from the on-axis target. To this end, a surgical apparatus 200 may output a laser beam 201 based on a set of therapy parameters designed to treat on-axis targets 32, while one or more scanners in the optics 500a, 500b may scan the laser beam 201 and/or the OCT beam 301 based on a set of scanning parameters designed to image and/or treat on-axis targets 32.

### Off-Axis to On-Axis Adaptive System

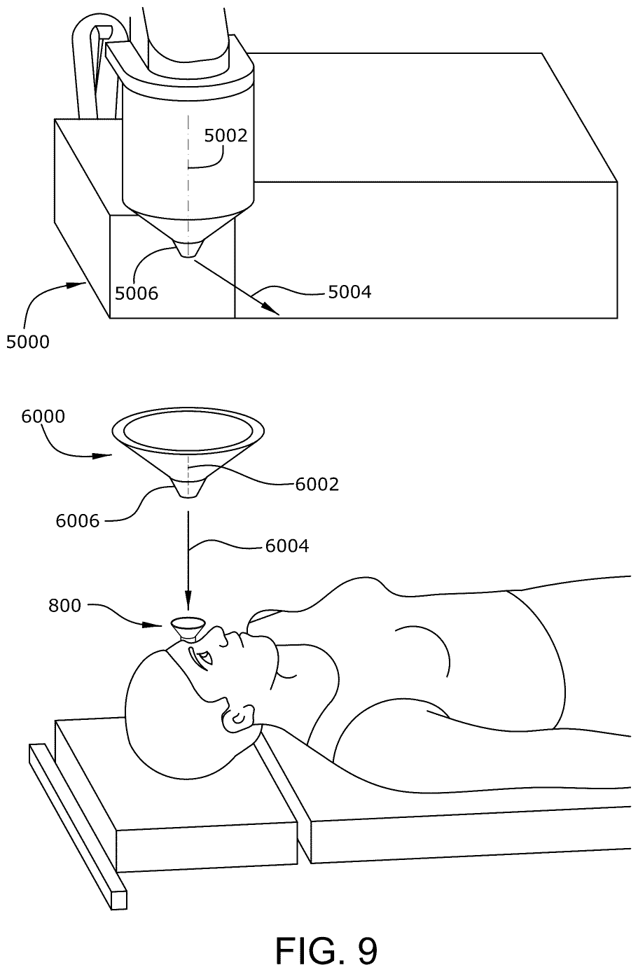

With reference to FIGS. 9, 3A, and 3B, an ophthalmic system 1000 adapted for conversion from an off-axis to an on-axis system includes an off-axis system 5000 having an off-axis-system axis 5002 and an on-axis adapter 6000 having an on-axis-adapter axis 6002. The off-axis system 5000 can be an ophthalmic surgical system designed for off-axis surgery, such as glaucoma surgery. The off-axis system 5000 includes a distal end 5006 and optics that are configured to provide an off-axis beam path 5004 that extends through the distal end can be aligned with an off-axis target structure, e.g., irido-corneal angle 13.

The on-axis adapter 6000 includes a distal end 6006 and optics. The on-axis adapter 6000 is configured to couple to and decouple from the off-axis system 5000. When coupled to the off-axis system 5000, the optics of the on-axis adapter 6000 are optically aligned with the off-axis beam path 5004. The optics of the on-axis adapter 6000 are configured to provide an on-axis beam path 6004 that extends through the distal end and can be aligned with an on-axis target structure, e.g., targets in the anterior segment of the eye such as the crystalline lens 4, the posterior capsule of the lens, the anterior capsule of the lens, the vitreous humor 10 or the retina 11. Thus, the on-axis adapter 6000 converts the off-axis system 5000 that accesses the irido-corneal angle 13 to a system that accesses the anterior segment of the eye.

The various aspects of this disclosure are provided to enable one of ordinary skill in the art to practice the present invention. Various modifications to exemplary embodiments presented throughout this disclosure will be readily apparent to those skilled in the art. Thus, the claims are not intended to be limited to the various aspects of this disclosure but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims.

1. An adaptable ophthalmic system comprising:
an on-axis system having an on-axis-system axis and comprising a distal end and optics, the optics of the on-axis system configured to provide an on-axis beam path that extends through the distal end of the on-axis system at an angle within an on-axis threshold angle of the on-axis-system axis; and
an off-axis adapter having an off-axis-adapter axis and comprising a distal end and optics, the off-axis adapter removably coupled with the on-axis system to substantially coaxially align the off-axis-adapter axis with the on-axis-system axis and to optically couple the on-axis beam path with the optics of the off-axis adapter, the optics of the off-axis adapter configured to redirect the on-axis beam path to optically align with an off-axis beam path that extends through the distal end of the off-axis adapter at an angle greater than an off-axis threshold angle from the on-axis-system axis.

2. The adaptable ophthalmic system of claim 1, wherein the on-axis threshold angle is in a range of 0 to 20 degrees.

3. The adaptable ophthalmic system of claim 1, wherein the off-axis threshold angle is in a range of 20 to 90 degrees.

4. The adaptable ophthalmic system of claim 1, wherein the optics of the off-axis adapter comprises redirecting optics.

5. The adaptable ophthalmic system of claim 4, wherein the redirecting optics comprise a first mirror optically aligned with the on-axis beam path and a second mirror optically coupled with the first mirror.

6. The adaptable ophthalmic system of claim 5, wherein the optics of the off-axis adapter further comprises a distal optical element at the distal end, the distal optical element optically coupled with the second mirror.

7. The adaptable ophthalmic system of claim 6, wherein the distal optical element is configured to optically couple with an eye to substantially coaxially align the on-axis-system axis and the off-axis-adapter axis with an optical axis of the eye, and to decouple from the eye.

8. The adaptable ophthalmic system of claim 1, wherein:
the optics of the on-axis system comprises an optical element; and
the optics of the off-axis adapter comprises a proximal optical element configured to optically couple with and decouple from the optical element of the on-axis system.

9. The adaptable ophthalmic system of claim 1, wherein:
the optics of the on-axis system comprises focusing optics configured to focus a light beam aligned with the on-axis beam path within a focal range from the distal end of the on-axis system; and
the optics of the off-axis adapter comprises a focus extender aligned to receive a first light beam from the on-axis system, the focus extender configured to focus the first light beam within a focal range from the distal end of the off-axis adapter.

10. The adaptable ophthalmic system of claim 1, wherein the on-axis system comprises an apparatus that outputs a light beam and the optics of the on-axis system comprises a scanner configured to scan the light beam.

11. The adaptable ophthalmic system of claim 1, wherein the on-axis system comprises a first apparatus that outputs a first light beam and a second apparatus that outputs a second light beam, and the optics of the on-axis system comprises a beam combiner configured to combine the first light beam and the second light beam into a combined beam.

12. The adaptable ophthalmic system of claim 11, wherein the first apparatus is a surgical apparatus, the first light beam is a laser beam, the second apparatus is an OCT imaging apparatus, and the second light beam is an OCT beam.

13. The adaptable ophthalmic system of claim 1, wherein the off-axis adapter comprises:
an OCT beam collimator coupled to receive an OCT beam from an OCT beam source; and
the optics comprises one or more mirrors optically coupled with the OCT beam collimator, the one or more mirrors configured to direct the OCT beam along a beam path that is radially offset from the on-axis-system axis and the off-axis-adapter axis.

14. The adaptable ophthalmic system of claim 13, wherein one of the one or more mirrors is configured to scan the OCT beam.

15. The adaptable ophthalmic system of claim 1, wherein the off-axis adapter comprises:
a dual aiming beam apparatus configured to output a first aiming beam and a second aiming beam; and
the optics comprise a beam combiner configured to combine the dual aiming beams and a first light beam and to direct the combined beams along the off-axis beam path.

16. The adaptable ophthalmic system of claim 1, wherein the off-axis beam path is a first off-axis beam path, and the off-axis adapter comprises a camera apparatus optically coupled with the optics to receive a visual beam along a second off-axis beam path that is at an angle greater than an off-axis threshold angle from the on-axis-system axis.

17. The adaptable ophthalmic system of claim 1, further comprising a rotation attachment configured to couple to and decouple from the on-axis system and to rotate the off-axis adapter or components thereof about the off-axis-adapter axis without rotating the on-axis system.

18. The adaptable ophthalmic system of claim 17, wherein the rotation attachment is further configured to attach to a patient interface and to rotate the off-axis adapter or components thereof about the off-axis-adapter axis without rotating the patient interface.

19. The adaptable ophthalmic system of claim 18, wherein the rotation attachment 4000 comprises a rotation mechanism configured to engage a housing of the off-axis adapter.

20. The adaptable ophthalmic system of claim 1, wherein the off-axis adapter is configured to mechanically couple to and decouple from the on-axis system.

21. The adaptable ophthalmic system of claim 1, further comprising a controller configured to control one or more of the optics of the on-axis system and the optics of the off-axis adapter.

22. A method of optically accessing an off-axis structure of an eye, the method comprising:
providing an on-axis beam path that extends through a distal end of an on-axis system having an on-axis-system axis, wherein the on-axis beam path is at an angle within an on-axis threshold angle of the on-axis-system axis; and
substantially coaxially aligning the on-axis-system axis with an off-axis-adapter axis of an off-axis adapter having optics configured to redirect the on-axis beam path to optically align with an off-axis beam path that extends through a distal end of the off-axis adapter, wherein the off-axis beam path is at an angle greater than an off-axis threshold angle from the on-axis-system axis.

23. The method of claim 22, wherein the on-axis threshold angle is in a range of 0 to 20 degrees.

24. The method of claim 22, wherein the off-axis threshold angle is in a range of 20 to 90 degrees.

25. The method of claim 22, wherein the off-axis adapter has an off-axis-adapter axis and optics, and optically aligning the on-axis beam path with the off-axis beam path comprises optically coupling the on-axis beam path with the optics.

26. The method of claim 25, wherein optically coupling the on-axis beam path with the optics comprises removably coupling the off-axis adapter to the on-axis system.

27. The method of claim 26, wherein the on-axis-system axis is substantially coaxially aligned with the off-axis-adapter axis.

28. The method of claim 22, further comprising rotating the off-axis adapter or components thereof about an off-axis-adapter axis without rotating the on-axis system.

29. The method of claim 22, further comprising rotating the off-axis adapter or optical components thereof about an off-axis-adapter axis without rotating a patient interface.

30. The method of claim 22, wherein the on-axis system comprises focusing optics configured to focus a light beam, the off-axis adapter comprises focusing optics configured to focus the light beam, and further comprising transferring the focus of the focusing optics of the on-axis system to a focus of the focusing optics of the off-axis adapter.

31. The method of claim 30, wherein transferring the focus of the light beam comprises extending the focus.

32. The method of claim 22, further comprising directing a light beam along the on-axis beam path and the off-axis beam path.

33. The method of claim 32, wherein the light beam comprises one of a laser beam, an OCT beam, a visual observation beam, and a dual aiming beam.

34. The method of claim 32, wherein the light beam is a combined beam, the method further comprising combining at least two of a laser beam, an OCT beam, a visual observation beam, and a dual aiming beam to provide the combined beam.

35. The method of claim 34, wherein the on-axis system comprises optics configured to provide the combined beam.

36. The method of claim 34, wherein the off-axis adapter comprises optics configured to provide the combined beam.

37. An off-axis adapter for directing a light beam between a distal end of an on-axis system and a structure distal the on-axis system, the on-axis system having an on-axis-system axis and comprising optics configured to provide an on-axis beam path that extends through the distal end at an angle within an on-axis threshold angle of the on-axis-system axis, the off-axis adapter comprising:
an off-axis-adapter axis;
a proximal optical element configured to optically couple to and decouple from the on-axis system, wherein the off-axis-adapter axis is substantially coaxially aligned with the on-axis-system axis when the proximal optical element is optically coupled to the on-axis system;
a distal optical element configured to optically couple to and decouple from the structure;
optics between the proximal optical element and the distal optical element, the optics optically aligned with the on-axis beam path and configured to convert the on-axis beam path to an off-axis beam path that extends through the distal optical element at an angle greater than an off-axis threshold angle from the on-axis-system axis.

38. The off-axis adapter of claim 37, further comprising:
an OCT beam collimator coupled to receive an OCT beam from an OCT beam source; and
the optics comprises one or more mirrors optically coupled with the OCT beam collimator, the one or more mirrors configured to direct the OCT beam along a beam path that is radially offset from the on-axis-system axis and the off-axis-adapter axis.

39. The off-axis adapter of claim 38, wherein one of the one or more mirrors is configured to scan the OCT beam.

40. The off-axis adapter of claim 37, further comprising:
a dual aiming beam apparatus configured to output a first aiming beam and a second aiming beam; and
the optics comprise a beam combiner configured to combine the dual aiming beams and a first light beam and to direct the combined beams along the off-axis beam path.

41. The off-axis adapter of claim 37, wherein the off-axis beam path is a first off-axis beam path, and further comprising a camera apparatus optically coupled with the optics to receive a visual beam along a second off-axis beam path that is at an angle greater than an off-axis threshold angle from the on-axis-system axis.

42. The off-axis adapter of claim 37, further comprising a rotation attachment configured to couple to and decouple from the on-axis system and to rotate the off-axis adapter about the off-axis-adapter axis without rotating the on-axis system.

43. The off-axis adapter of claim 42, wherein the rotation attachment is further configured to attach to a patient interface and to rotate the off-axis adapter about the off-axis-adapter axis without rotating the patient interface.

44. The off-axis adapter of claim 43, wherein the rotation attachment comprises a rotation mechanism configured to engage a housing of the off-axis adapter.

45. An adaptable ophthalmic system comprising:
a system having a system axis and comprising optics coupled to receive a light beam and configured to direct the light beam along a first beam path; and
an adapter having an adapter axis, the adapter configured to removably couple with the system to substantially coaxially align the adapter axis with the system axis when coupled to the system, and comprising optics optically coupled with the optics of the system to receive the light beam from the system along the first beam path and configured to redirect the light beam to a second beam path that is angularly offset from the first beam path by a threshold angle.

46. The adaptable ophthalmic system of claim 45, wherein:
the system is an on-axis system and the first beam path is an on-axis beam path that extends through a distal end of the on-axis system, wherein the on-axis beam path is at an angle within an on-axis threshold angle of the system axis; and
the adapter is an off-axis adapter and the second beam path is an off-axis beam path that extends through a distal end of the off-axis adapter, wherein the off-axis beam path is at an angle greater than an off-axis threshold angle from the system axis.

47. The adaptable ophthalmic system of claim 45, wherein:
the system is an off-axis system and the first beam path is an off-axis beam path that extends through a distal end of the off-axis system, wherein the off-axis beam path is at an angle greater than an off-axis threshold angle from the system axis; and
the adapter is an on-axis adapter and the second beam path is an on-axis beam path that extends through a distal end of the on-axis adapter, wherein the on-axis beam path is at an angle within an on-axis threshold angle of the system axis.
